SEQUENCE LISTING (12 sequences)

SEQ ID NO: 1 - protein, 5 aa, human
NSGMN

SEQ ID NO: 2 - protein, 17 aa, human
WINTYNGEPTYTDDFKG

SEQ ID NO: 3 - protein, 11 aa, human
HSYGSSYAMDN

SEQ ID NO: 4 - protein, 15 aa, human
KASQSVDYDGDSYMN

SEQ ID NO: 5 - protein, 7 aa, human
VASNLKS

SEQ ID NO: 6 - protein, 9 aa, human
QQSNEEPWT

SEQ ID NO: 7 - protein, 120 aa, human
QIQLVQSGPELKKPGASVKVSCRASGYPFTNSGMNWVKQAPGKGLKWMGWINTYNGEPTYTDDFKGRFAFSLETSASTAYLQINNLRNEDTATYFCASHSYGSSYAMDNWGQGTSVTVSS

SEQ ID NO: 8 - protein, 111 aa, human
DIVLTQSPDSLAVSLGERATINCKASQSVDYDGDSYMNWYQQKPGQPPKLLIYVASNLKSGIPARFSGSGSGTDFTLTISSLQPEDFATYYCQQSNEEPWTFGGGTKVEIK

SEQ ID NO: 9 - DNA, 360 bp, human
cagattcagctggtgcagtccggccctgagctgaagaaacctggcgcctccgtgaaggtgtcctgcagagcttccggctaccccttcaccaactccggcatgaactgggtcaagcaggcccctggcaagggcctgaagtggatgggctggatcaacacctacaacggcgagcccacctacaccgacgacttcaagggcagattcgccttctccctggaaacctccgcctccaccgcctacctgcagatcaacaacctgcggaacgaggacaccgccacctacttttgcgcctcccactcctacggctccagctacgccatggacaattggggccagggcacctccgtgaccgtgtctagc

SEQ ID NO: 10 - DNA, 333 bp, human
gatatcgtgctgacccagtcccctgactccctggctgtgtctctgggcgagagagccaccatcaactgcaaggcctcccagtccgtggactacgacggcgactcctacatgaactggtatcagcagaagcccggccagccccccaagctgctgatctacgtggcctccaacctgaagtccggcatccctgccagattctccggctctggctctggcaccgactttaccctgaccatctccagcctgcagcccgaggacttcgccacctactactgccagcagtccaacgaggaaccctggacctttggcggaggcaccaaggtggaaatcaag

SEQ ID NO: 11 - protein, 450 aa, human
QIQLVQSGPELKKPGASVKVSCRASGYPFTNSGMNWVKQAPGKGLKWMGWINTYNGEPTYTDDFKGRFAFSLETSASTAYLQINNLRNEDTATYFCASHSYGSSYAMDNWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 218 aa, human
DIVLTQSPDSLAVSLGERATINCKASQSVDYDGDSYMNWYQQKPGQPPKLLIYVASNLKSGIPARFSGSGSGTDFTLTISSLQPEDFATYYCQQSNEEPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0192836 A1
(43) Pub. Date: Jun. 22, 2023

(54) CCL24 INHIBITORS IN THE TREATMENT OF COVID-19

(71) Applicant: CHEMOMAB LTD., Tel Aviv (IL)

(72) Inventors: Adi MOR, Tel Aviv (IL); Arnon AHARON, Tel Aviv (IL); Michal SEGAL SALTO, Tel Aviv (IL); Neta BARASHI, Tel Aviv (IL)

(73) Assignee: CHEMOMAB LTD., Tel Aviv (IL)

(21) Appl. No.: 17/996,120

(22) PCT Filed: Apr. 20, 2021
(86) PCT No.: PCT/IL2021/050446

§ 371 Date: Oct. 13, 2022

Related U.S. Application Data
(60) Provisional application No. 62/704,079, filed on Apr. 20, 2020.

(51) Int. Cl. C07K 16/24

(52) U.S. Cl. CPC C07K 16/24; C07K 2317/24

(57) ABSTRACT

The invention concerns isolated monoclonal antibodies that bind to CCL24 (eotaxin 2) for use in the treatment of Covid-19.

### TECHNOLOGICAL FIELD

The present invention concerns the use of inhibitors of CCL24 (Eotaxin-2), in particular anti CCL24 antibodies, in the treatment of Covid-19.

### BACKGROUND ART

References considered to be relevant as background to the presently disclosed subject matter are listed below:

1. Heiman et al. (2005) Cytokine-stimulated human lung alveolar epithelial cells release eotaxin-2 (CCL24) and eotaxin-3 (CCL26). Journal of Interferon and Cytokine Research 25:82-91.

2. Huang et al. (2020) Clinical features of patients infected with 2019 novel coronavirus in Wuhan, China. Lancet, 395, 497-506.

3. Kohan et al. (2010) Eotaxin-2/CCL24 and eotaxin-3/CCL26 exert differential profibrogenic effects on human lung fibroblasts. Ann Allergy Asthma Immunol. 104:66 ---72.

4. Mor et al. (2019) Blockade of CCL24 with a monoclonal antibody ameliorates experimental dermal and pulmonary fibrosis. Ann Rheum Dis 0:1-9.

5. Shi, Y., Y. Wang, C. Shao, J. Huang, J. Gan, X. Huang, E. Bucci, M. Piacentini, G. Ippolito & G. Melino (2020) COVID-19 infection: the perspectives on immune responses. Cell Death & Differ 27 (1451-1454).

6. Zhang, J. J., X. Dong, Y. Y. Cao, Y. D. Yuan, Y. B. Yang, Y. Q. Yan, C. A. Akdis & Y. D. Gao (2020) Clinical characteristics of 140 patients infected with SARS-CoV-2 in Wuhan, China. Allergy. Volume 75, Issue 7, 1730-1741.

Acknowledgement of the above references herein is not to be inferred as meaning that these are in any way relevant to the patentability of the presently disclosed subject matter.

### BACKGROUND

The severe acute respiratory syndrome coronavirus 2 (SARS-COV2) is a newly identified member of the coronavirus family. This virus was first identified in Wuhan, China and was announced as a world pandemic by the World Health Organization (WHO) named COVID-19 (coronavirus disease 2019). The virus represents a strain of RNA viruses that has not been previously identified in humans and could induce symptoms including fever, dry cough, dyspnea, fatigue, and lymphopenia in infected patients. In more severe cases, infections causing viral pneumonia characterized primarily by fever, cough, dyspnea and bilateral infiltrates may lead to severe acute respiratory syndrome (SARS) and even death (Zhang et al. 2020). When COVID-19 infects the upper and lower respiratory tract it can cause mild or highly acute respiratory syndrome with consequent release of pro-inflammatory cytokines and chemokines. The virus can also result in a dysregulated immune response and this cytokine storm seems to be associated with disease severity, as it can lead to capillary leak syndrome, progressive lung injury, respiratory failure and acute respiratory distress syndrome (ARDS) (Zhang et al. 2020; Huang et al. 2020; Shi et al. 2020).

It was found that CCL24 promotes lung damage through two main pathways: activation of lung fibroblast cells and immune cells recruitment (Kohan et al. 2010, Heiman et al. 2005, Mor et al. 2019).

WO 2015/132790 discloses polyspecific antibodies directed to a unique epitope in the chemokine eotaxin 2 (termed CM-101), whereby the antibodies bind additional CCR3-binding chemokines. WO 2015/132790 further concerns use of these antibodies for attenuating the migration of various cells and for treating fibrotic diseases, autoimmune inflammatory disorders, monocyte related disorders or allergic atopic disorders. WO 2018/163185 discloses the use of anti CCL24 antibodies for treating hepatic fibrotic disorders. CM-101 was also shown to inhibit collagen secretion in the lung and reduce immune cell recruitment to the lung (Mor et al. 2019).

None of the above publications, or any other in the art, showed increased levels of CCL24 in Covid-19 patients or suggested that inhibition of CCL24 may have positive implications on the treatment of Covid-19.

### GENERAL DESCRIPTION

In a first of its aspects the present invention provides an isolated monoclonal antibody, or any antigen-binding fragment thereof, that binds CCL24 for use in the treatment of Covid-19.

In some embodiments, said monoclonal antibody is a chimeric antibody, a human antibody, a humanized antibody or a fully humanized antibody.

In some embodiments said antigen-binding fragment thereof is selected from the group consisting of Fv, single chain Fv (scFv), heavy chain variable region capable of binding the antigen, light chain variable region capable of binding the antigen, Fab, F(ab)₂’ and any combination thereof.

In a specific embodiment, said antibody is a fully humanized antibody comprising a heavy chain variable region comprising:
a) the complementary determining region VH CDR1 comprising the amino acid sequence denoted by SEQ ID NO. 1 or a variant thereof;
b) the complementary determining region VH CDR2 comprising the amino acid sequence denoted by SEQ ID NO. 2 or a variant thereof; and
c) the complementary determining region VH CDR3 comprising the amino acid sequence denoted by SEQ ID NO. 3 or a variant thereof; and a light chain variable region comprising
d) the complementary determining region VK CDR1 comprising the amino acid sequence denoted by SEQ ID NO. 4 or a variant thereof;
e) the complementary determining region VK CDR2 comprising the amino acid sequence denoted by SEQ ID NO. 5 or a variant thereof; and
f) the complementary determining region VK CDR3 comprising the amino acid sequence denoted by SEQ ID NO. 6 or a variant thereof.

In one embodiment, said antibody is a fully humanized antibody comprising the heavy chain variable region denoted by SEQ ID NO:7 or a variant thereof and the light chain variable region denoted by SEQ ID NO: 8 or a variant thereof.

In another aspect, the present invention provides a pharmaceutical composition comprising a fully humanized antibody comprising a heavy chain variable region comprising:
a) the complementary determining region VH CDR1 comprising the amino acid sequence denoted by SEQ ID NO. 1 or a variant thereof;
b) the complementary determining region VH CDR2 comprising the amino acid sequence denoted by SEQ ID NO. 2 or a variant thereof; and
c) the complementary determining region VH CDR3 comprising the amino acid sequence denoted by SEQ ID NO. 3 or a variant thereof; and a light chain variable region comprising
d) the complementary determining region VK CDR1 comprising the amino acid sequence denoted by SEQ ID NO. 4 or a variant thereof;
e) the complementary determining region VK CDR2 comprising the amino acid sequence denoted by SEQ ID NO. 5 or a variant thereof; and
f) the complementary determining region VK CDR3 comprising the amino acid sequence denoted by SEQ ID NO. 6 or a variant thereof;
and a pharmaceutically acceptable carrier; wherein said pharmaceutical composition is for use in the treatment of COVID-19.

In another aspect, the present invention provides a method of treating COVID-19 comprising administering to a patient in need thereof a therapeutically acceptable amount of a fully humanized antibody comprising a heavy chain variable region comprising:
a) the complementary determining region VH CDR1 comprising the amino acid sequence denoted by SEQ ID NO. 1 or a variant thereof;
b) the complementary determining region VH CDR2 comprising the amino acid sequence denoted by SEQ ID NO. 2 or a variant thereof; and
c) the complementary determining region VH CDR3 comprising the amino acid sequence denoted by SEQ ID NO. 3 or a variant thereof; and a light chain variable region comprising
d) the complementary determining region VK CDR1 comprising the amino acid sequence denoted by SEQ ID NO. 4 or a variant thereof;
e) the complementary determining region VK CDR2 comprising the amino acid sequence denoted by SEQ ID NO. 5 or a variant thereof; and
f) the complementary determining region VK CDR3 comprising the amino acid sequence denoted by SEQ ID NO. 6 or a variant thereof, or a pharmaceutical composition comprising said antibody.

### DETAILED DESCRIPTION OF EMBODIMENTS

The present invention is based on the concept that an antibody that is directed to CCL24 (Eotaxin 2) and which binds and inhibits the activity of eotaxin 2 is useful in the treatment of Covid-19 patients with pneumonia. The fully humanized monoclonal antibody (termed herein CM101) targets the soluble chemokine CCL24 and prevents it from stimulating its receptor.

In vitro and in vivo studies have been conducted to evaluate the activation of CCR3 expressing cells in the presence of CCL24 and CM 101. In vitro data has demonstrated that CM-101 causes a significant reduction of CCL24 dependent immune cell recruitment. CM-101 attenuated the activation of CCR3 expressing cells, including inflammatory and fibrotic cells expressing CCR3 involved in the development of lung fibrosis (WO 2015/132790, WO 2018/163185)

Blocking CCL24 using CM-101 was shown to substantially attenuate fibrosis and inflammation across a wide range of in vitro and in vivo models, including experimental idiopathic pulmonary fibrosis and systemic sclerosis models where a significant amelioration of the lung disease was detected following treatments with CM-101 ((WO 2015/132790).

The presented invention suggests that CM-101 has the potential to attenuate CCL24 induced activities including migration and activation of immune cells and lung fibroblast while maintaining a favorable safety profile, therefore is suggested as a therapy to patients that have a severe lung damage due to COVID-19.

CM101 has been implicated previously for use in the treatment of fibrotic diseases, hepatic disorders, autoimmune inflammatory disorders, monocyte related disorders, or allergic atopic disorders. The present invention provides for the first time a novel use for the antibody in the treatment of Covid-19 patients that suffer from lung damage.

CCR3 (C-C chemokine receptor type 3) is a protein that in humans is encoded by the CCR3 gene. CCR3 has also recently been designated CD193 (cluster of differentiation 193). The protein encoded by this gene is a receptor for C-C type chemokines. It is a 7-transmembrane G protein-coupled receptor which is expressed by eosinophils as well as by a wide array of cell types including macrophages and endothelial cells. This receptor binds and responds to a variety of chemokines, including eotaxin (also termed eotaxin 1 or CCL11), eotaxin-2 (CCL24), eotaxin-3 (CCL26), MCP-3 (CCL7), MCP-4 (CCL13), and RANTES (CCL5).

The terms “eotaxin 2” (eosinophil chemotactic protein 2), “CCL24” (Chemokine (C-C motif) ligand 24) or “MPIF-2” (myeloid progenitor inhibitory factor 2) are used interchangeably and refer to a cytokine belonging to the CC chemokine family which is encoded by the human CCL24 gene, located on human chromosome 7. CCL24 interacts with chemokine receptor CCR3. CCL24 activity includes induction of chemotaxis in eosinophils, basophils, T lymphocytes and neutrophils, as well as induction of angiogenic and migratory responses in endothelial and smooth muscle cells.

The present invention thus provides an isolated monoclonal antibody, or any antigen-binding fragment thereof, that binds to CCL24 for use in the treatment of Covid-19.

The terms “Covid 19” and “COVID-19” are used interchangeably herein to denote a medical condition characterized by a viral infection caused by the severe acute respiratory syndrome coronavirus 2 (SARS-COV2).

As indicated above, the present invention provides isolated monoclonal antibodies that bind to CCL24 chemokine for use in the treatment of Covid-19.

The term “antibody” refers to a polypeptide encoded by an immunoglobulin gene or functional fragments thereof that specifically bind and recognize an antigen, namely proinflammatory CCR3-binding chemokines. Specifically, the antibody of the invention binds and recognizes at least eotaxin 2, eotaxin 1, RANTES and MCP-3.

In a preferred embodiment the antibody of the invention is a monoclonal antibody. The term “monoclonal antibody”, “monoclonal antibodies” or “mAb” as herein defined refers to a population of substantially homogenous antibodies, i.e., the individual antibodies comprising the population are identical except for possibly naturally occurring mutations that may be present in minor amounts. Monoclonal antibodies are directed against a single antigenic site.

Monoclonal antibodies may be prepared and purified by any method known in the art. For example, monoclonal antibodies may be prepared from B cells taken from the spleen or lymph nodes of immunized animals (e.g. rats or mice), by fusion with immortalized B cells under conditions which favor the growth of hybrid cells.

Immunization of mice may be carried out for example as described in WO 2010/086854. Briefly, immunization of mice may be carried out for example by primary sub-cutaneous (s.c.) immunization with the desired antigen, namely with a CCR3- binding chemokine, e.g. eotaxin 2, or with a fragment of a CCR3-binding chemokine comprising a conformational epitope in the N-loop (e.g. 50 µg) emulsified with complete Freund’s emulsified with incomplete Freund’s adjuvant are then administered every 2 weeks. The mice with the highest neutralizing antibody titer receive an additional intravenous (i.v.) boost of the antigen (e.g. 5 µg) in PBS four days prior to spleen removal.

After the final boost (e.g., four days), the spleen of the mouse with the highest neutralizing antibody titer is removed and splenocytes are fused to mouse myeloma cells (e.g., NS0 cells) using polyethylene glycol, as previously described (Köhler, G. and Milstein, C. (1975) Nature 256: 495-497). After fusion, the hybridoma cells are selected by growing the cells in HAT (hypoxantine-aminopterin-thymidine) medium. Cell clones are then screened for specific antibody production, for example using the ELISA assays described below.

Purification of monoclonal antibodies may be based for example on affinity chromatography, namely, using an affinity column to which the specific epitope is conjugated.

An exemplary antibody structural unit comprises a tetramer, as known in the art. Each tetramer is composed of two identical pairs of polypeptide chains, each pair having one “light chain” and one “heavy chain”. The N-terminus of each chain defines a variable region of about 100 to 110 or more amino acids primarily responsible for antigen (or epitope) recognition.

Thus, the terms “heavy chain variable region” (V_{H}) and “light chain variable region” (V_{L}) refer to these heavy and light chains, respectively. More specifically, the variable region is subdivided into hypervariable and framework (FR) regions. Hypervariable regions have a high ratio of different amino acids in a given position, relative to the most common amino acid in that position. Four FR regions which have more stable amino acids sequences separate the hypervariable regions. The hypervariable regions directly contact a portion of the antigen’s surface. For this reason, hypervariable regions are herein referred to as “complementarity determining regions”, or “CDRs”.

From N-terminal to C-terminal, both light and heavy chains comprise the domains FR1, CDR1, FR2, CDR2, FR3, CDR3 and FR4. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the specific CDR is located.

Thus, the complementarity determining regions CDRH1, CDRH2 and CDRH3 refer to the three complementarity determining regions starting from the N-terminus of the antibody’s heavy chain and the complementarity determining regions CDRL1, CDRL2 and CDRL3 refer to the three complementarity determining regions starting from the N-terminus of the antibody’s light chain.

In some embodiments the isolated poly-specific monoclonal antibody is a chimeric antibody, a human antibody, a humanized antibody or a fully humanized antibody.

The term “chimeric” antibodies as herein defined refers to antibodies in which a portion of the heavy and/or light chain is derived from a particular species, while the remainder of the chain(s) is derived from another species, as well as fragments of such antibodies, which exhibit the same biological activity.

Chimeric antibodies may be prepared by any method known in the art, for example as described below.

A murine-human chimeric antibody may be prepared by the amplification and cloning of murine V_{H} and V_{L} genes, encoding the antibody variable regions, followed by murine-human chimeric antibody expression. To this end, total RNA is isolated from murine anti-eotaxin 2 hybridoma cells that are shown to secrete antibodies with the desired characteristics and cDNA is synthesized using oligo (dT)₁₅ primer, M-MLV and AMV reverse transcriptases. Amplification of the heavy and the light variable genes (V_{H} and V_{L}) may be carried out using a panel of primers directed at the 5′ terminus of framework 1 of each gene, essentially as described in Benhar and Reiter (Benhar, I. and Reiter, Y. (2002) Curr. Protoc. Immunol. Chapter 10: Unit 10 19B), and to the constant region (C_{H}1 or Cₖ, respectively) at the 3′ end.

The variable genes are then re-amplified using non-degenerate primers introducing restriction sites at both ends for cloning, for example, into a pCMV-based antibody expression vector.

The amplified heavy and light variable genes are separately purified, digested and cloned into appropriate mammalian full-length Ig expression vectors, providing each chain with a corresponding signal-peptide and constant gene, resulting in IgG1/k murine human chimeric antibody expression. For preparing large quantities of the antibody, a stable cell line expressing the antibody can be prepared, by transfecting cells (e.g., CHO cells) with the Ig expression vector containing both heavy and light chains of the chimeric antibody. Highly producing clones may be then selected and expanded based on antibody levels in the supernatant, as tested by any method known in the art, for example, a CCR3-binding chemokine-specific ELISA assay, as detailed herein below.

The term “humanized” antibodies traditionally refers to forms of non-human (for example, murine) antibodies that contain a human-derived immunoglobulin framework with minimal sequences derived from non-human immunoglobulin at the CDRs and optionally at additional relevant positions. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and activity.

As used herein the term “fully humanized” antibodies relates to antibodies designed to have only human sequences. The fully humanized antibodies of the present invention were prepared using the Composite Human Antibodies™ technology that minimizes immunogenicity of the antibodies in patients. In this humanization technology multiple sequence segments derived from variable (V) regions of unrelated human antibodies are used as acceptors for the complementarity determining regions (CDRs) of the starting antibodies. Through careful selection of human sequence segments and the application of in silico tools, CD4+ T cell epitopes are avoided so the risk of immunogenicity is reduced compared to standard humanized antibodies whilst antibody affinity and specificity is maintained. Such antibodies contain only human sequences and are thus defined as “fully humanized”.

The term “human antibody” as used herein refers to an antibody that possesses an amino acid sequence corresponding to that of an antibody produced by a human and/or has been made using any of the techniques for making human antibodies known in the art. This definition specifically excludes a humanized antibody that comprises non-human antigen-binding residues.

Preparation of humanized and human antibodies is well known in the art. Antibodies may also be prepared using phage display. As known in the art, antibody phage display (APD) is based on genetic engineering of bacteriophages and repeated rounds of antigen-guided selection and phage propagation.

The APD process begins with antibody-library preparation, by preparation of quality RNA from the cell source chosen (e.g., peripheral blood mononuclear cells). This RNA is reverse-transcribed into cDNA, which is used for PCR of the VH and VL chains of the encoded antibodies. This step is followed by ligation of the variable heavy (VH) and variable light (VL) PCR products into a phage display vector, culminating in analysis of clones of mAbs.

In some embodiments the present invention provides an isolated monoclonal antibody, or any antigen-binding fragment thereof, that binds to CCL24 for use in the treatment of Covid-19, wherein said antibody is a fully humanized antibody and comprises a heavy chain variable region comprising:
a) the complementary determining region VH CDR1 comprising the amino acid sequence denoted by SEQ ID NO. 1 (NSGMN) or a variant thereof;
b) the complementary determining region VH CDR2 comprising the amino acid sequence denoted by SEQ ID NO. 2 (WINTYNGEPTYTDDFKG) or a variant thereof; and
c) the complementary determining region VH CDR3 comprising the amino acid sequence denoted by SEQ ID NO. 3 (HSYGSSYAMDN) or a variant thereof; and
a light chain variable region comprising
d) the complementary determining region VK CDR1 comprising the amino acid sequence denoted by SEQ ID NO. 4 (KASQSVDYDGDSYMN) or a variant thereof;
e) the complementary determining region VK CDR2 comprising the amino acid sequence denoted by SEQ ID NO. 5 (VASNLKS) or a variant thereof; and
f) the complementary determining region VK CDR3 comprising the amino acid sequence denoted by SEQ ID NO. 6 (QQSNEEPWT) or a variant thereof.

The above CDR sequences CDRH1, CDRH2, CDRH3, CDRL1, CDRL2 and CDRL3 denoted by SEQ ID NO. 1, SEQ ID NO. 2, SEQ ID NO. 3, SEQ ID NO. 4, SEQ ID NO. 5 and SEQ ID NO. 6, respectively are also presented in the context of their respective heavy and light chains sequences:

The amino acid sequence of the heavy chain variable region of the isolated fully humanized poly-specific monoclonal antibody exemplified herein is denoted by SEQ ID NO: 7 and is of the amino acid sequence:

QIQLVQSGPELKKPGASVKVSCRASGYPFTNSGMNWVKQAPGKGLKWMGW
INTYNGEPTYTDDFKGRFAFSLETSASTAYLQINNLRNEDTATYFCASHS
YGSSYAMDNWGQGTSVTVSS

The amino acid sequence of the light chain variable region of the isolated humanized poly- specific monoclonal antibody exemplified herein is denoted herein by SEQ ID NO: 8 and is of the amino acid sequence:

DIVLTQSPDSLAVSLGERATINCKASQSVDYDGDSYMNWYQQKPGQPPKL
LIYVASNLKSGIPARFSGSGSGTDFTLTISSLQPEDFATYYCQQSNEEPW
TFGGGTKVEIK

Therefore, in further embodiments the isolated antibody for use according to the invention is wherein said antibody is a fully humanized antibody comprising the heavy chain variable region denoted by SEQ ID NO:7 or a variant thereof and the light chain variable region denoted by SEQ ID NO: 8 or a variant thereof.

The present invention also encompasses variants of the heavy and light chain variable regions. The variants may include mutations in the complementarity determining regions of the heavy and light chains which do not alter the activity of the antibodies herein described, or in the framework region.

By the term “variant” it is meant sequences of amino acids or nucleotides different from the sequences specifically identified herein, in which one or more amino acid residues or nucleotides are deleted, substituted or added.

It should be appreciated that by the term “added”, as used herein it is meant any addition of amino acid residues to the sequences described herein.

Variants encompass various amino acid substitutions. An amino acid “substitution” is the result of replacing one amino acid with another amino acid which has similar or different structural and/or chemical properties. Amino acid substitutions may be made on the basis of similarity in polarity, charge, solubility, hydrophobicity, hydrophilicity, and/or the amphipathic nature of the residues involved.

Typically, variants encompass conservative amino acid substitutions. Conservative substitution tables providing functionally similar amino acids are well known in the art. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

Conservative nucleic acid substitutions are nucleic acid substitutions resulting in conservative amino acid substitutions as defined above.

Variants in accordance with the invention also encompass non-polar to polar amino acid substitutions and vice-versa.

As used herein, the term “amino acid” or “amino acid residue” refers to naturally occurring and synthetic amino acids, as well as amino acid analogs and amino acid mimetics that function in a manner similar to the naturally occurring amino acids.

Variant sequences refer to amino acid or nucleic acids sequences that may be characterized by the percentage of the identity of their amino acid or nucleotide sequences with the amino acid or nucleotide sequences described herein (for example, the amino acid or nucleotide sequences of the heavy and light chains of the antibodies herein described).

In some embodiments, variant sequences as herein defined refer to nucleic acid sequences that encode the heavy and light chain variable regions, each having a sequence of nucleotides with at least 70% or 75% of sequence identity, around 80% or 85% of sequence identity, around 90%, 91%, 92%, 93%, 94%, 95%, 96%, 97%, 98% or 99% of sequence identity when compared to the sequences of the heavy and light chain variable regions described herein.

In one embodiment, the nucleic acid sequence encoding the heavy chain variable region of the isolated fully humanized poly-specific monoclonal antibody exemplified herein is denoted by SEQ ID NO: 9 and is of the nucleic acid sequence:

CAGATTCAGCTGGTGCAGTCCGGCCCTGAGCTGAAGAAACCTGGCGCCTC
CGTGAAGGTGTCCTGCAGAGCTTCCGGCTACCCCTTCACCAACTCCGGCA
TGAACTGGGTCAAGCAGGCCCCTGGCAAGGGCCTGAAGTGGATGGGCTGG
ATCAACACCTACAACGGCGAGCCCACCTACACCGACGACTTCAAGGGCAG
ATTCGCCTTCTCCCTGGAAACCTCCGCCTCCACCGCCTACCTGCAGATCA
ACAACCTGCGGAACGAGGACACCGCCACCTACTTTTGCGCCTCCCACTCC
TACGGCTCCAGCTACGCCATGGACAATTGGGGCCAGGGCACCTCCGTGAC
CGTGTCTAGC

In one embodiment, the nucleic acid sequence encoding the light chain variable region of the isolated humanized poly- specific monoclonal antibody exemplified herein is denoted herein by SEQ ID NO: 10 and is of the nucleic acid sequence:

GATATCGTGCTGACCCAGTCCCCTGACTCCCTGGCTGTGTCTCTGGGCGA
GAGAGCCACCATCAACTGCAAGGCCTCCCAGTCCGTGGACTACGACGGCG
ACTCCTACATGAACTGGTATCAGCAGAAGCCCGGCCAGCCCCCCAAGCTG
CTGATCTACGTGGCCTCCAACCTGAAGTCCGGCATCCCTGCCAGATTCTC
CGGCTCTGGCTCTGGCACCGACTTTACCCTGACCATCTCCAGCCTGCAGC
CCGAGGACTTCGCCACCTACTACTGCCAGCAGTCCAACGAGGAACCCTGG
ACCTTTGGCGGAGGCACCAAGGTGGAAATCAAG

Therefore, in further embodiments the isolated antibody for use according to the invention is wherein said antibody is a fully humanized antibody comprising the heavy chain variable region denoted by SEQ ID NO:7 or a variant thereof and the light chain variable region denoted by SEQ ID NO: 8 or a variant thereof.

The amino acid sequence of the heavy chain of the isolated fully humanized monoclonal antibody exemplified herein is denoted by SEQ ID NO: 11. The amino acid sequence of the light chain of the isolated fully humanized monoclonal antibody exemplified herein is denoted by SEQ ID NO: 12.

Therefore, in further embodiments the isolated antibody for use according to the invention is wherein said antibody is a fully humanized antibody comprising the heavy chain denoted by SEQ ID NO: 11 or a variant thereof and the light chain denoted by SEQ ID NO: 12 or a variant thereof.

By the term “activity of the antibodies” it is meant the ability of the antibodies to bind to at least two CCR3-binding chemokines, and preferably to inhibit a biological function mediated by such CCR3-binding chemokines.

Non-limiting examples of such biological functions are: inhibition of cell recruitment or chemotaxis (for example recruitment or chemotaxis of eosinophils or monocytes or fibroblasts), inhibition of the transition of fibroblasts to myoblasts, or reduction of cellular activation (for example as measured by Ca⁺ update). The biological functions can be measured in vivo or in vitro using methods well known in the art. Several such assays are described in the Examples below.

Additional in vitro experiments for determining the binding of the antibody prepared according to the invention to its target protein include for example ELISA assays.

The present invention further encompasses any antigen-binding fragments of the isolated monoclonal antibody of the invention. Such antigen-binding fragments may be for example Fab and F (ab′)₂, which are capable of binding antigen. Such fragments may be produced by any method known in the art, for example by proteolytic cleavage, using enzymes such as papain (to produce Fab fragments) or pepsin (to produce F(ab′)₂ fragments).

Thus in some embodiments the isolated monoclonal antibody according to the invention is wherein said antibody is an antibody fragment selected from the group consisting of Fv, single chain Fv (scFv), heavy chain variable region capable of binding the antigen, light chain variable region capable of binding the antigen, Fab, F(ab)_{2′} and any combination thereof.

In another one of its aspects the present invention provides an isolated nucleic acid molecule comprising a nucleotide sequence encoding an antibody or any antigen-binding fragment thereof according to the invention.

The term “nucleic acid” or “nucleic acid molecule” as herein defined refers to a polymer of nucleotides, which may be either single- or double-stranded, which is a polynucleotide such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, single-stranded (such as sense or antisense) and double- stranded polynucleotides. The term DNA used herein also encompasses cDNA, i.e. complementary or copy DNA produced from an RNA template by the action of reverse transcriptase (RNA-dependent DNA polymerase).

The invention further provides an expression vector comprising the isolated nucleic acid molecule as herein defined.

“Expression vector” sometimes referred to as “expression vehicle” or “expression construct”, as used herein, encompasses vectors such as plasmids, viruses, bacteriophage, integratable DNA fragments, and other vehicles, which enable the integration of DNA fragments into the genome of the host. Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. The expression vector in accordance with the invention may be competent with expression in bacterial, yeast, or mammalian host cells, to name but few.

In yet another one of its aspects the present invention provides a host cell transfected with the isolated nucleic acid molecule according to the invention or with the expression vector according to the invention.

The term “host cells” as used herein refers to cells which are susceptible to the introduction of the isolated nucleic acid molecule according to the invention or with the expression vector according to the invention. Preferably, said cells are mammalian cells, for example CHO cells or NS0 cells. Transfection of the isolated nucleic acid molecule or the expression vector according to the invention to the host cell may be performed by any method known in the art.

In yet another one of its aspects the present invention provides an immunoconjugate comprising the antibody or any antigen-binding fragment thereof according to the invention and an additional therapeutic agent, e.g. an anti-inflammatory agent.

The term “immunoconjugate” as herein defined refers to an antibody or any antigen-binding fragment thereof according to the invention that is conjugated (linked or joined) to an additional agent. Immunoconjugates may be prepared by any method known to a person skilled in the art, for example, by cross-linking the additional agent to the antibody according to the invention or by recombinant DNA methods.

The antibody of the invention may be administered in combination with at least one additional therapeutic agent.

The term “additional therapeutic agent” used herein refers to any agent that may be used for treating FMS. In accordance with certain embodiments said at least one additional therapeutic agent is selected from a group consisting of chemotherapeutics, cytokines, peptides, antibodies and antibiotics.

In certain embodiments, said additional therapeutic agent includes, but is not limited to pain relievers (e.g. Acetamirophen, Ibuprofen, Naproxen sodium, Tramadol), Antidepressants (e.g. Duloxetine, Venlafaxine, Milnacipran, Amitriptyline, Fluoxetine, Paroxetine), Anti-seizure drugs (e.g. Gabapentin and Pregabalin) and muscle relaxants (e.g. Diazepam).

In certain embodiments the additional therapeutic agent is an additional antibody. The term “additional antibody” as herein defined refers to an antibody, which is not the antibody according to the invention, which may be used in combination with the antibody of the invention.

The present invention further provides a pharmaceutical composition comprising as an active ingredient the isolated antibody of the invention, or any antigen-binding fragment thereof or the immunoconjugate as herein defined and a pharmaceutically acceptable carrier, excipient or diluents, wherein said pharmaceutical composition is for use in the treatment of COVID-19.

The “pharmaceutical composition” of the invention generally comprises the antibody or any antigen-binding fragment thereof as herein defined and a buffering agent, an agent which adjusts the osmolarity of the composition and optionally, one or more pharmaceutically acceptable carriers, excipients and/or diluents as known in the art.

As used herein the term “pharmaceutically acceptable carrier, excipient or diluent” includes any and all solvents, dispersion media, coatings, antibacterial and antifungal agents and the like, as known in the art. The carrier can be solvent or dispersion medium containing, for example, water, ethanol, polyol (for example, glycerol, propylene glycol, and liquid polyethylene glycol, and the like), suitable mixtures thereof, and vegetable oils. Each carrier should be both pharmaceutically and physiologically acceptable in the sense of being compatible with the other ingredients and not injurious to the subject. Except as any conventional media or agent is incompatible with the active ingredient, its use in the therapeutic composition is contemplated.

In specific embodiments the present invention relates to a pharmaceutical composition comprising an isolated monoclonal humanized antibody, or any antigen-binding fragment thereof, which binds to CCL24, wherein the antibody comprises a heavy chain variable region of the amino acid sequence denoted by SEQ ID NO. 7 or a variant thereof and a light chain variable region of the amino acid sequence denoted by SEQ ID NO. 8, or a variant thereof, wherein said pharmaceutical composition is for use in the treatment of Covid-19.

In some embodiments the invention provides a method of treatment or amelioration of Covid-19, comprising administering to a subject in need thereof a therapeutically effective amount of an isolated monoclonal humanized antibody, or any antigen-binding fragment thereof, which binds to CCL24, wherein the antibody comprises a heavy chain variable region of the amino acid sequence denoted by SEQ ID NO. 7 or a variant thereof and a light chain variable region of the amino acid sequence denoted by SEQ ID NO. 8, or a variant thereof.

The terms “subject” or “patient” are used interchangeably and refer to a subject that may benefit from the present invention such as a mammal (e.g., canine, feline, ovine, porcine, equine, bovine, or human). In one specific embodiment the patient is human.

It is to be understood that the terms “treat”, “treating”, “treatment” or forms thereof, as used herein, mean reducing, preventing, curing, reversing, ameliorating, attenuating, alleviating, minimizing, suppressing or halting the deleterious effects of a disease or a condition or delaying the onset of one or more clinical indications of Covid-19. In an embodiment, a clinical indication of Covid-19 is a severe acute respiratory syndrome (SARS) or pneumonia.

Administration according to the present invention may be performed by any of the following routes: oral administration, intravenous, intramuscular, intraperitoneal, intratechal or subcutaneous injection; intrarectal administration; intranasal administration, ocular administration or topical administration.

In specific embodiments administration according to the present invention may be performed intravenously. In other specific embodiments administration may be performed intraperitoneally. In other specific embodiments administration may be performed by inhalation.

The antibodies or antibody fragments as herein defined, any pharmaceutical compositions comprising the same or any conjugates comprising them may be administered to a subject prior to or post disease onset.

Thus, in some embodiments the method of treatment or amelioration of Covid-19 according to the invention is where said isolated humanized monoclonal antibody or any antigen-binding fragment thereof according to the invention, or pharmaceutical composition according to the invention is administered to said subject prior to or after disease onset.

A “therapeutically effective amount” of the isolated monoclonal antibody or any antigen-binding fragment thereof according to the invention, or the pharmaceutical composition according to the invention for purposes herein defined is determined by such considerations as are known in the art in order to cure, arrest or at least alleviate or ameliorate the medical condition. For any preparation used in the methods of the invention, the dosage or the therapeutically effective amount can be estimated initially from in vitro cell culture assays or based on animal models such as the animal models detailed herein.

In some embodiments the therapeutically effective amount in accordance with the invention is in the range of 0.01 to 100 mg/kg.

In other embodiments the therapeutically effective amount in accordance with the invention is in the range of 0.01 to 40 mg/kg, 0.1 to 40 mg/kg, 1 to 10 mg/kg, or 5 to 10 mg/kg.

In other embodiments the isolated humanized monoclonal antibody or any antigen-binding fragment thereof according to the invention or pharmaceutical composition according to the invention is administered to the subject as a single dose or multiple doses.

The term “subject in need thereof” in the context of the present invention refers to mammals and in particular to human subjects suffering from Covid-19.

The present invention further provides the isolated humanized monoclonal antibody or any antigen-binding fragment thereof according to the invention or the pharmaceutical composition according to the invention for use in a method of prophylaxis, treatment or amelioration of Covid-19.

In specific embodiments the invention provides an isolated humanized monoclonal antibody, or any antigen-binding fragment thereof, which binds to CCL24, wherein the antibody comprises a heavy chain variable region of the amino acid sequence denoted by SEQ ID NO. 7 or a variant thereof and a light chain variable region of the amino acid sequence denoted by SEQ ID NO. 8, or a variant thereof for use in a method of prophylaxis, treatment or amelioration of FMS.

It is appreciated that the term purified or “isolated” refers to molecules, such as amino acid or nucleic acid sequences, peptides, polypeptides or antibodies that are removed from their natural environment, isolated or separated. An “isolated antibody” is therefore a purified antibody. As used herein, the term “purified” or “to purify” also refers to the removal of contaminants from a sample.

### EXAMPLES

### Determination of Chemokine Serum Profile

This study is performed in order to determine the chemokine and inflammatory mediator serum profile of patients with COVID-19 related pneumonia. This study enables the elucidation of a possible link between inflammatory mediators and the severity of the COVID-19 related pneumonia.

### Patients Inclusion Criteria

Patients at the age of ≥ 18, with diagnosis of SARS-CoV-2 infection using PCR and confirmation of COVID-19 related pneumonia by imaging or clinical presentation.

### Preparation of Samples

Whole blood samples are obtained and kept at room temperature for 30 min. The whole blood samples are then centrifuged at 3000 rpm for 15 minutes. The serum is deposited into a new tube and can be kept frozen at -80° C. or stored at 4° C. for subsequent analysis.

### Cytokine Analysis

Levels of CCL24, as well as other inflammatory mediators are determined in the sera, saliva or bronchoalveolar lavage (BAL) fluid of the Covid-19 patients using the following Elisa kit: Quantikine CCL24/Eotaxin-2, (R&D), according to the manufacturer’s protocol.

### ELISA

The ELISA is performed according to the manufacturer’s instructions in the relevant kit. Briefly, 100 µL of each tested sample and a standard (present in the appropriate kit) are placed in appropriate wells. The samples are incubated for 2.5 hours at room temperature or over-night at 4° C. with gentle shaking. The solution is discarded and the well is washed 4 times with 300 µL 1X Wash Solution. After the last wash, any remaining 1X Wash Buffer is removed by aspirating or decanting. Next, 100 µL, of the detection antibody (for example, 1X Biotinylated Eotaxin-2 Detection Antibody) is added to each well and incubated for 1 hour at room temperature with gentle shaking. The solution is discarded and the wells are washed 4 times with the Wash solution as described above. 100 µL of 1X HRP-Streptavidin solution is added to each well and incubate for 45 minutes at room temperature with gentle shaking. The solution is discarded and the wells are washed as noted above. Next, 100 µL, of TMB One-Step Substrate Reagent is added to each well and incubated for 30 minutes at room temperature in the dark with gentle shaking. Finally, 50 µL of Stop Solution are added to each well. The results are read at 450 nm immediately.

### Administration of CM101 Antibody to Covid-19 Patients

Covid-19 patients with pneumonia recruited as described above are treated with a single dose of CM-101. The patients are then followed-up for three weeks. In case of clinical benefit, a second dose is optionally administered. Patients are treated with the antibody at a dose range of 5-10 mg/kg.

1-7. (canceled)

8. A method of treating Covid-19 comprising administering to a patient in need thereof a therapeutically acceptable amount of an isolated monoclonal antibody, or any antigen-binding fragment thereof, that binds to CCL24.

9. The method of claim 8 wherein said monoclonal antibody is a chimeric antibody, a human antibody, a humanized antibody, or a fully humanized antibody.

10. The method of claim 8 wherein said antigen-binding fragment thereof is selected from the group consisting of Fv, single chain Fv (scFv), heavy chain variable region capable of binding the antigen, light chain variable region capable of binding the antigen, Fab, F(ab)2′ and any combination thereof.

11. The method of claim 8, wherein said antibody is a fully humanized antibody comprising a heavy chain variable region comprising:
a) the complementary determining region VH CDR1 comprising the amino acid sequence denoted by SEQ ID NO. 1 or a variant thereof;
b) the complementary determining region VH CDR2 comprising the amino acid sequence denoted by SEQ ID NO. 2 or a variant thereof; and
c) the complementary determining region VH CDR3 comprising the amino acid sequence denoted by SEQ ID NO. 3 or a variant thereof; and a light chain variable region comprising
d) the complementary determining region VK CDR1 comprising the amino acid sequence denoted by SEQ ID NO. 4 or a variant thereof;
e) the complementary determining region VK CDR2 comprising the amino acid sequence denoted by SEQ ID NO. 5 or a variant thereof; and
f) the complementary determining region VK CDR3 comprising the amino acid sequence denoted by SEQ ID NO. 6 or a variant thereof.

12. The method of claim 8, wherein said antibody is a fully humanized antibody comprising the heavy chain variable region denoted by SEQ ID NO:7 or a variant thereof and the light chain variable region denoted by SEQ ID NO: 8 or a variant thereof.
